(19) United States
(12) Patent Application Publication
(10) Pub. No.: US 2019/0175507 A1
(43) Pub. Date: Jun. 13, 2019

(54) COMPOSITIONS FOR ORAL ADMINISTRATION OF ACTIVE AGENTS

(71) Applicant: Tamarisk Technologies Group LLC, Moorpark, CA (US)

(72) Inventors: Claudia M. CARDONA, Danville, VA (US); Mark Ennis KETNER, Fuquay Varina, NC (US); Dakshinamurthy Devanga CHINTA, Cary, NC (US); Carl R. SAHI, Coventry, CT (US); John M. POLIDORO, Tolland, CT (US); James-Thomas Marion GLADDEN, Raleigh, NC (US)

(21) Appl. No.: 16/306,220

(22) PCT Filed: Jun. 2, 2017
(86) PCT No.: PCT/US17/35659

§ 371 Date: Nov. 30, 2018

Related U.S. Application Data
(60) Provisional application No. 62/345,411, filed on Jun. 3, 2016.

(51) Int. Cl. A61K 9/16; A61K 45/00; A61K 9/00; A61K 9/19

(52) U.S. Cl. CPC A61K 9/1694; A61K 45/00; A61K 9/0053; A61K 9/1623; A61K 9/1647; A61K 9/1635; A61K 9/1652; A61K 9/19

(57) ABSTRACT

Described herein are microparticles suitable of oral administration of active agents to a subject in need thereof. The microparticles will localize in the gastrointestinal tract of the subject without crossing the intestinal mucosa into the intestinal bloodstream. The microparticles are resistant to degradation in the stomach, and can deliver the active agent to the intestinal lumen, while the microparticle itself is excreted by the subjects system. The microparticles are self-sustaining bodies of a crosslinked polymer matrix and an active agent encapsulated therein. The polymer matrix further comprises a plurality of delivery enhancing moieties, wherein at least a portion of the delivery enhancing moieties being presented on the exterior surface of the microparticle.

### CROSS-REFERENCE TO RELATED APPLICATIONS

The present application claims the priority benefit of U.S. Provisional Patent Application Ser. No. 62/345,411, filed Jun. 3, 2016, entitled COMPOSITIONS FOR ORAL ADMINISTRATION OF ACTIVE AGENTS, incorporated by reference in its entirety herein.

### BACKGROUND OF THE INVENTION

### Field of the Invention

The present invention relates generally to compositions of and methods for the manufacturing and use of effective drug delivery formulations.

### Description of Related Art

Many pharmaceutical and nutraceutical products are unsuitable for oral ingestion. Barriers to oral administration of these therapeutic agents include, but are not limited to, a need to carefully regulate or target dosage, an inability to readily absorb these agents from the gastrointestinal (GI) tract, the denaturation and digestion of protein drugs by gastric juices, or the requirement for hydrophobic/hydrophilic stabilizers in some compositions.

However, oral ingestion is generally viewed by most clinicians as the ideal method of delivery for lowering the equally important barrier of patient compliance. Therefore, there exists an unmet need for delivery systems capable of overcoming these and other similar challenges.

### SUMMARY OF THE INVENTION

The invention is broadly concerned with drug delivery particles, and particularly the modification of various swellable and/or biodegradable polymers for the purpose of improving the stability of active agents within the particle as well as, for example, in the presence of the concentrated acid of gastric juices, and the uptake of these agents by appropriate target cells. The invention further describes delivery platforms engineered for the administration of pharmaceuticals by non-invasive routes such as oral, buccal, rectal, ocular, transdermal, intravaginal, pulmonary, and nasal routes.

In one or more embodiments, described herein are microparticles particularly suited for oral delivery of active agents to a subject in need thereof. The microparticles comprise a self-sustaining body having an exterior surface. The self-sustaining body comprises a crosslinked polymer matrix and an active agent encapsulated therein, along with optional time-release excipients. The active agent is distributed (preferably substantially uniformly) throughout the polymer matrix, which further comprises a plurality of delivery enhancing moieties, wherein at least a portion of the delivery enhancing moieties are presented on the exterior surface of the self-sustaining body. Advantageously, the microparticle is resistant to enteric degradation and will localize in the GI tract of the subject. The microparticle is also mucoadhesive, and will temporarily adhere to the epithelial lining of the GI tract, without crossing the intestinal mucosa into the intestinal bloodstream. Rather, the microparticle will swell in the small intestine, thereby releasing its active agent payload to be taken up into the bloodstream, while the remainder of the particle itself will be cleared and excreted through normal GI excretion mechanisms.

The particle formulations and methods contemplated herein are suitable for the encapsulation and subsequent oral, or other mucosal, delivery of a broad spectrum of hydrophobic and hydrophilic biologically active, therapeutic, or nutritionally-useful active agents such as, but not limited to, small molecules, peptides, proteins, lipids, carbohydrates, nucleic acids, cyclic peptides, monoclonal antibodies, antibiotics, antiviral agents, anti-inflammatory agents, anti-tumor agents, polypeptides, steroidal agents, anti-sense agent, RNA agents, DNA agents, carotenoids, vitamins, minerals, phototropic agents, and anthocyanins.

Therapeutic and prophylactic compositions are also described herein. The compositions generally comprise a therapeutically-effective amount of the microparticles according to the various described embodiments, dispersed in a pharmaceutically acceptable carrier. Such compositions can be in unit dosage form, and particularly, unit dosage forms suitable for oral administration (e.g., capsule, tablet, powder, liquid suspension, etc.).

Methods of administering an active agent to a subject in need thereof are also contemplated herein. The methods generally comprise administering a therapeutically effective amount of microparticles or compositions according to the various described embodiments to the subject. In general, the microparticles or compositions will be orally administered. It will be appreciated that “oral administration” also includes prescribing such active agents to the subject, where such microparticles are compositions are subsequently self-administered (e.g., ingested) by the subject.

Also described herein are uses of a composition according to the various described embodiments to administer a therapeutically effective amount of active agent to a subject in need thereof.

The present disclosure is also concerned with novel approaches for forming microparticles for drug delivery, such that the microparticles will localize in the gastrointestinal tract, without crossing the intestinal mucosa into the intestinal bloodstream. The methods generally comprise providing a polymer suspension comprising a polymer matrix precursor (i.e., the polymer before crosslinking), and delivery enhancing moieties, dispersed in a solvent system. The polymer suspension is combined with an active agent (or active agent suspension) to yield a mixture. The polymer matrix precursor is then crosslinked in the mixture to yield a crosslinked polymer matrix in the form of a self-sustaining body having an exterior surface, wherein the active agent is distributed throughout the polymer matrix, and wherein at least a portion of the delivery enhancing moieties are presented on the exterior surface of the self-sustaining body. Embodiments of the invention include generating droplets of the mixture, and further rapid freezing of such droplets prior to crosslinking.

Additional embodiments of the invention are described in more detail below.

### BRIEF DESCRIPTION OF THE DRAWINGS

FIG. 1 provides structural representations of exemplary carotenoids.

FIG. 2 is a depiction of one embodiment of a carotenoid-bound alginate polymer.

FIG. 3 provides the structural representation of alginate.

FIG. 4 is a representation of an agarobiose disaccharide.

FIG. 5 is a representation of a polymer suspension.

FIG. 6A is a representation of an aqueous polymer suspension after it has been mixed with a delivery enhancing moiety.

FIG. 6B is a representation of the polymer suspension mixture with active agent.

FIG. 7 represents a solid/crosslinked microparticle with active agent.

FIG. 8 is an illustration of delivery enhancing moiety-bound polymers being recognized and adhered to the outer surface of mucosal enterocytes of the small intestine.

FIG. 9A is a micrograph of the inventive microparticles adhering to an epithelial cell.

FIG. 9B is a micrograph of an epithelial cell with a particle lacking the delivery enhancing moiety.

FIG. 10A is a graph of data for the TA-RU animals receiving unencapsulated active agent.

FIG. 10B is a graph of plasma Concentrations (ng/mL) of Resveratrol in Orally Dosed Sprague-Dawley rats with 355 or 395 mg/kg invention encapsulations 1-3.

FIG. 11 shows blood plasma resveratrol concentrations at various dosing per animal.

### DETAILED DESCRIPTION

The present invention is broadly concerned with microparticles particularly suited for oral delivery of active agents. In particular, the microparticles have improved stability in the presence of concentrated acid, and facilitate improved release and uptake of the active agent in the GI tract when administered orally. Embodiments of the invention concern the use of delivery enhancing moieties as well as excipients which regulate the time-release of active agent. Methods of forming uniformly-sized microparticles are also described herein, involving a novel coacervation procedure. These particles can hold active agents uniformly distributed within the polymer matrix. The invention further describes engineering of the polymer particles for mucosal delivery of the active agents, and in particular, for the oral delivery route.

The microparticles are discrete, self-sustaining bodies, each comprising a cured or crosslinked polymer matrix and a delivery enhancing moiety. Exemplary polymers that can be used include polyvinyl alcohol, poly(ethylene glycol), polylactic acid, poly-L-lactic acid, polycaprolactone, polyglycolic acid, poly(lactic-co-glycolic acid), polyhydroxyalkanoate, poly [N-(2-hydroxypropyl) methacrylamide], hyaluronic acid, gelatin, cellulose derivatives, and polysaccharides such as xanthan gum, chitosan, alginate, and/or pectin. Combinations of the foregoing polymers can also be used. In one or more embodiments, the polymer matrix comprises (consists essentially or even consists entirely of) swellable and/or biodegradable polymers such as sodium, potassium, or calcium alginate, and optionally contains another polymer such as, but not limited to, carrageenan, xanthan gum, agar-agar, and/or chitosan.

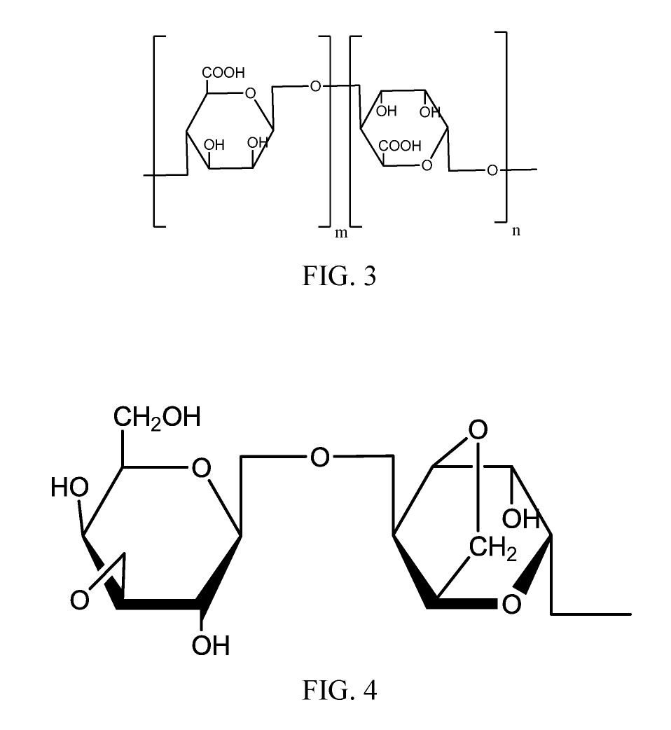

Sodium alginate (and potassium alginate) has the unusual ability to form a gel upon agitation within cold water which will not solidify upon standing. The gels thus formed have a high encapsulation affinity, meaning the ability of the alginate molecule to surround and wind itself around another molecule. In this way a three-dimensional network builds up in which double helices form junction points of the polymer chains thus allowing for the formation of multiple helix-helix aggregates which wind around and encapsulate the active agent. Sodium alginate is typically obtained by extraction from brown algae and is widely used within the food industry to increase product viscosity and as an emulsifying agent. Sodium alginate has an empirical formula of NaC₆ H₇O₆ having a molecular structure as shown in FIG. 3. Alginates are linear unbranched polymers containing (1→4)-linked D-mannuronic acid (M) and its epimer α-(1→4)-linked L-guluronic acid (G). D-mannuronic acid residues are enzymatically converted to L-guluronic after polymerization.

Alginates are not random copolymers but, according to the algal source, comprise blocks of similar alternating residues, each of which have different conformational preferences and behavior. The alginate polymer may comprise, for example, homopolymeric blocks of consecutive G-residues, or consecutive M-residues, or alternating M- and G-residues or randomly organized blocks of G- and M-residues. For example, the M/G ratio of alginate from Macrocystis pyrifera is about 1.6, whereas that from Laminaria hyperborea is about 0.45.

Although sodium alginate (or potassium alginate) in itself is very effective in molecular encapsulation activity, an even higher encapsulation affinity to the active agent therein can be obtained, if desired, through the addition of 0.1 to 1% to 2% to 3% to 4% to 5% or more of a thickening agent or cross-linking agent such as carrageenan, xanthan gum, and/or agar-agar to the alginate. Agar-agar is extracted from the cell membranes of some species of red algae, particularly those from the genera Gelidium and Gracilaria. Historically agar-agar has chiefly been used as an ingredient in desserts, especially in Japan agar-agar comprises a mixture of agarose and agaropectin. Agarose is a linear polymer, of molecular weight about 120,000, based on the (1→3)-β-D-galactopyranose-(1→4)-3, 6-anhydro-α-L-galactopyranose unit. Agaropectin is a heterogeneous mixture of smaller molecules that occur in lesser amounts. Their structures are similar but slightly branched and sulfated, and they may have methyl and pyruvic acid ketal substituents. The molecular structure of agarobiose disaccharide units is shown in FIG. 4.

Modification of the polymers used in the microparticles includes the incorporation of selected delivery enhancing moieties which interact with the polymers directly in ionic, covalent, or supramolecular manners or indirectly via physical entrapment within the polymer matrix. Delivery-enhancing moieties used to modify the polymers include molecules capable of facilitating the absorption and uptake of active agents by cells located throughout the GI tract and the subsequent systemic bioavailability of these agents. The delivery enhancing moieties can be first dispersed in a suitable aqueous- or oil-based carrier to facilitate even distribution of the delivery enhancing moieties upon combining with the polymer. Exemplary pharmaceutically acceptable carriers for use in the particles are described in more detail below.

In one or more embodiments, delivery enhancing moieties are selected from the group consisting of (1) medium or long chain fatty acids, (2) isoprenoids, (3) vitamins, (4) signal peptides, and combinations thereof. The incorporation of the delivery enhancing moieties into the chosen swellable and/or biodegradable polymers can be achieved through ionic, covalent, or supramolecular bonds as appropriate according to the respective functional groups of the polymer and the delivery enhancing moieties. The delivery enhancing moieties can be covalently linked to the polymer. Alternatively, the delivery enhancing moieties can be associated with the polymer through non-covalent attachments (e.g., ionic bonds, van der Waals interactions, and the like). Other embodiments of the invention include the molecular association of delivery enhancing moieties with the swellable and/or biodegradable polymer through covalent or non-covalent bonding via functional groups other than hydroxyl groups (e.g., amines). The delivery enhancing moieties may also simply be physically restrained by the polymer matrix due to entrapment therein. It will be appreciated that a combination of approaches can be used to associate the delivery enhancing moieties with the polymer, which may depend on the particular polymer as well as the delivery enhancing moiety utilized in the particles.

Delivery enhancing moieties for use according to the various embodiments include long and medium chain fatty acids which generally have a chain length varying from 6-28 carbon atoms. For use herein, long chain fatty acids, especially fusogenic lipids (unsaturated fatty acids and monoglycerides such as oleic acid, linolenic acid, linoleic acid, monoolein, phosphatidylserine, and phosphatidylethanolamine) provide useful carriers to enhance targeted delivery and uptake of the active agents contemplated herein. Medium chain fatty acids (C₆ to C₁₂) and may also be used to enhance targeted delivery and uptake of the particle or payload encapsulated thereby. Other medium and long chain fatty acids that can be used as delivery enhancers herein include, but are not limited to myristoleic acid, palmitoleic acid, oleic acid, alpha-linolenic acid, arachidonic acid, eicosapentaenoic acid, erucic acid, and docosahexaenoic acid. Examples of naturally-occurring fatty acids which may be used in the presently claimed and disclosed inventive concept(s) include but are not limited to C8:0 (caprylic acid), C10:0 (capric acid), C12:0 (lauric acid), C14:0 (myristic acid), C16:0 (palmitic acid), C16:1 (palmitoleic acid), C16:2, C18: 0 (stearic acid), C18: 1 (oleic acid), C18: 1-7 (vaccenic), C18: 2-6 (linoleic acid), C18:3-3 (alpha-linolenic acid), C18:3-5 (eleostearic), C18:3-6 (delta-linolenic acid), C18:4-3, C20:1 (gondoic acid), C20: 2-6, C20:3-6 (dihomo-gamma-linolenic acid), C20:4-3, C20:4-6 (arachidonic acid), C20:5-3 (eicosapentaenoic acid), C22: 1 (docosenoic acid), C22:4-6 (docosatetraenoic acid), C22: 5-6 (docosapentaenoic acid), C22:5-3 (docosapentaenoic), C22:6-3 (docosahexaenoic acid) and C24: 1-9 (nervonic). Highly preferred unbranched, naturally occurring fatty acids are those with from 14 to 22 carbon atoms. In addition, sodium salts of medium and long chain fatty acids are effective delivery enhancing molecules.

Examples of isoprenoid-type delivery enhancing moieties used herein include, but are not limited to, lycopene, limonene, gamma-tocotrienol, geraniol, carvone, farnesol, geranylgeraniol, squalene, and other linear terpenoids, carotenoids, taxol, vitamin E, vitamin A, beta-carotene, Coenzyme Q10 (ubiquinone), astaxanthin, zeaxanthin, lutein, citranxanthin, beta-chloro-carotene, canthaxanthin, and modified forms thereof. Within the GI, a number of these delivery enhancing moieties interact with, at least, the scavenger receptor class B type I.

In one or more embodiments, where the delivery enhancing moiety is a carotenoid, the carotenoid hydroxyl group can be esterified with the carboxylic acid unit of an amino acid (—COOH). Then the amino group of the amino acid (—NHR) is coupled to the carboxylic acid units of the polymer matrix (—COOH). The amino acid can be either proteinogenic or non-proteinogenic in any stereochemical form. Amino acids used in this reaction include, but are not limited to, Glycine, Alanine, Valine, Leucine, Isoleucine, Serine, Cysteine, Selenocysteine, Threonine, Methionine, Proline, Phenylalanine, Tyrosine, Tryptophan, Histidine, Lysine, Arginine, Aspartate, Glutamate, Asparagine, and Glutamine. The carboxylic acid group of the amino acid is activated via an anhydride, acylimidazole, and acyl halide, such as acyl fluoride, acyl bromide, acyl chloride (by oxalyl chloride, thionyl chloride, phosphorus trichloride, phosphorus oxychloride, phosphorus pentachloride, cyanuric chloride), or via an activated ester formed by N-(3-Dimethylaminopropyl)-N′-ethylcarbodiimide hydrochloride (EDCI), N,N′-Diisopropylcarbodiimide (DIC), or N,N′-Dicyclohexylcarbodiimide (DCC), and 4-(Dimethylamino)pyridine DMAP. The ester bond can also be formed through other methods such as catalysis by cobalt(II) chloride hexahydrate.

The amino group (—NHR) of the amino acid, as well as any functional group on the side chain which can interfere with the ester formation, needs to be protected. The amino groups can be protected (—NR′R, where R′ is the protecting group) with tert-butoxycarbonyl groups (t-Boc), or 9-fluorenylmethoxycarbonyl (Fmoc), or any other protecting groups strategically chosen in an orthogonal manner for the specific amino acid so that there is not interference in attaining the desired ester.

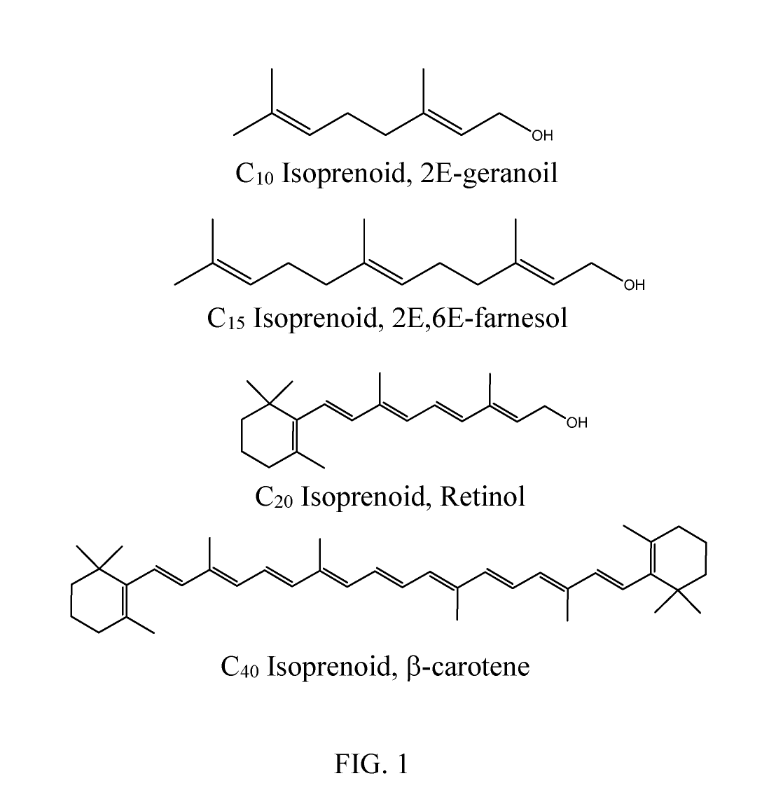

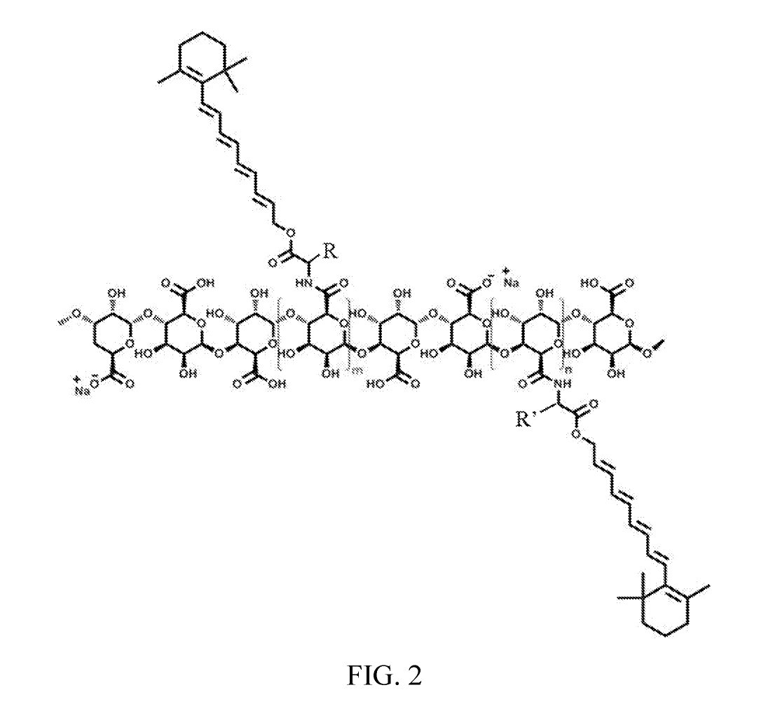

After the esterification reaction reaches completion, the amino group (—NR′R) is deprotected to be available to react with the carboxylic acid groups (—COOH) of the polymer, such as alginate, in aqueous media. The latter reaction can be conducted through chemoenzymatic protocols, or with the activation of the carboxylic acid groups of the polymer with EDCI and N-Hydroxysuccinimide or 1-Hydroxy-2,5-pyrrolidinedione NHS resulting in the carotenoids covalently attached to the polymer separated by an amino acid linker. In various embodiments of the invention, the carotenoid can encompass any of available hydroxylated-carotenes such as, but not limited to, retinol, lutein, zeaxanthin, cryptoxanthin, hydroxyechinenone, astaxanthine, diatoxanthin, dinoxanthin, antheraxantin, diadinoxanthin, echinenone, neoxanthin, flavoxanthin, violaxanthine, rubixanthin, fucoxanthin, and isomers thereof. See FIG. 1 for an illustration of example compounds. FIG. 2 illustrates by way of non-limiting example, alginate polymer modified by the covalent attachment of retinol.

Delivery enhancing moieties contemplated herein also include vitamins, as well as signal peptides.

The microparticles can also include additives to regulate the time-release profile of the active agent. Excipients regulating time-release (referred to herein a “time-release excipients”) interact with the polymers and/or the active agent in a manner causes the components of the particle to dissociate/diffuse from the microparticle differently with respect to time. Examples of time-release excipients which can be used in the presently claimed and disclosed inventive concept(s) include, but are not limited to, polar molecules such as polyhydroxyl molecules, which include monosaccharides (glucose, fructose, galactose, xylose, mannose, tagatose), disaccharides (trehalose, lactose, sucrose, maltose, isomaltose, trehalulose), sugar alcohols (erythritol, glycerol, isomalt, lactitol, maltitol, mannitol, sorbitol, xylitol), polyoxy molecules (polyethylene glycols (PEGs), Sorbitan, Tween, Polysorbate, poloxamers), and/or chitosan.

The particle used in the presently claimed and disclosed inventive concept(s) may also contain small quantities of butylated hydroxy toluene, glycerine, polyethylene glycols, propylene glycol, lecithin, antioxidants, tocopherol, docosahexaenoic acid, and pirotiodecane in addition to coloring agents, solubilizers, and extenders.

One or more active agents can be entrapped in the polymer matrix and thereby be encapsulated in the microparticle. The polymer matrix is able to encapsulate many classes or types of active agents (aka active pharmaceutical ingredients, API) including, but not limited to, therapeutic or nutraceutical agents, such as antibiotics, antivirals, antioxidants, oncological agents, anti-lipids, antihypertensives, cardiac drugs, antidiabetic agents, vitamins, minerals, proteins, peptidomimics, microorganisms, monoclonal antibodies, and/or RNA or DNA molecules. More specifically the encapsulated active agent of the presently claimed and disclosed inventive concept(s) may be selected, for example, from anabolic agents (e.g., boldandiol, ethylestrenol, mibolerone, nandrolone, oxymetholone, stanozol, and testosterone); antibacterial/antibiotics (e.g., aminoglycosides including: amikacin, apramycin, dihydrostreptomycin, gentamicin, kanamycin, neomycin, spectinomycin, vancomycin; cephalosporins including: cefaclor, ceftazidime, cephalexin, cephalothin; clindamycin; chlorhexidine, fatty acid monoesters, such as glycerol monolaurate; fluoroquinolones including enroflaxacin, ciprofloxacin; macrolides including erythromycin, lincomycin, tylosin; penicillins including amoxicillin with and without potentiators, ampicillin, hetacillin, ticarcillin; tetracycline and analogues; sulfonomides with or without potentiators including sulfachloropyridazine, sulfadimethoxine, sulfamethazine, and sulfaquinoxaline); antifungals (e.g., miconazole, itraconazole, griseofulvin, glycerol mono-laureate, and metronidazole); anti-cancer agents (e.g., actinomycin-D, cisplatin, cytarabine, doxorubicin, 5-fluorouracil, methotrexate, pergolide, purine analogues, oncovin, vinblastine, and vincristine); antidotes and reversing agents (e.g., atropine, 2-PAM, naloxone, and nalorphine HCI, yohimbine, (atipamezole); antihistamines (e.g., cromolyn sodium, diphenhydramine, pyrilamine, and tripelennamine); antipyretics (e.g. acetaminophen); non-steroidal anti-inflammatory drugs (NSAIDs), (e.g., flunixin meglumine, acetylsalicylic acid, ibuprofen, ketoprofen, meclofenamic acid, naproxen, phenylbutazone, and zileuton); steroidal anti-inflammatory drugs (e.g., beclomethasone, budesonide, dexamethasone, flumethasone, flunisolide, fluticasone, isoflupredone, prednisolone, and triamcinolone); anti-thrombotics (e.g., acetylsalicylic acid); anti-tussives (e.g., narcotic analgesics, dextromethorphan, and pholcodine); bronchodilators (e.g., atropine, albuterol, clenbuterol, pirbuterol, salmeterol, fenoterol, aminophylline, glycopyrrolate, terbutaline, and theophylline); parasympathomimetics (e.g., bethanechol); anticholinergics (e.g., atropine, ipratropium, and tiotropium); anti-virals (e.g. pyrimidine nucleosides including idoxuridine, and trifluridine; purine nucleosides including: vidarabine, and acyclovir; ribavirin, amantadine, interferon and its inducers, and other miscellaneous anti-virals, for example, thiosemicarbazones, zidovudine, and benzimidazoles); sympathomimetics (e.g., epinephrine); cardiovascular agents (e.g., calcium channel blockers: diltiazem, nifedipine, and verapamil); anti-arrhythmics (e.g., alprenolol, amiodarone, bretylium, diltiazem, flecainide, isoproterenol, lidocaine, metoprolol, nadolol, procainamide, propranolol, quinidine, timolol, and verapamil); vasoactive drugs (e.g., captopril, epinephrine, hydralazine, isoxsuprine, nitroglycerin, pentoxifylline, phentolamine, and prazosin); cardiotonics (e.g., dobutamine; dopamine; digitoxin; and digoxin); central nervous agents: e.g., anesthetics including barbiturates; anticonvulsants e.g., clonazepam, diphenylhydantoin, primidone; antidepressants: e.g., SSRI (selective serotonin re-uptake inhibitor); antiemetics: e.g., domperidone, metoclopramide; emetics: apomorphine; narcotic analgesics: codeine, demerol, fentanyl, hydrocodone, meperidine, morphine, oxymorphone, butorphanol, buprenorphine, pentazocine; non-narcotic analgesics including acetaminophen, aspirin, dipyrone; respiratory stimulants: e.g., caffeine, doxapram, zolazepam; sedatives/tranquilizers including: barbiturates; alpha 2 antagonists (e.g., detomidine, medetomidine, dexmedetomidine, carfentanil, diazepam, droperidol, ketamine, midazolam, phenothiazine tranquilizers (including acepromazine, chlorpromazine, ethylisobutrazine, promazine, and triflupromazine), romifidine, xylazine; diuretics (e.g., chlorothiazide, and furosemide); dental hygiene (e.g., glycerol monolaurate materials and orally active antibiotics); gastrointestinal agent (e.g., cimetidine (H2 agonist), famotidine, ranitidine, and omeprazole); hypotensives (e.g., acepromazine, and phenoxybenzamine); hormones (e.g., ACTH, altrenogest, estradiol 17beta, estrogens GNRH, FSH, LH, insulin, LHRH, megestrol, melatonin, misoprostol, norgestomet, progesterone, testosterone, thyroxine, and trenbolone); immunomodulators (stimulants including: levamisole, imiquimod and analogues, biological derivative products; and suppressants including: azathioprine); internal parasiticides (e.g., ivermectin, mebendazole, monensin, morantel, moxidectin, oxfendazole, piperazine, praziquantel, and thiabendazole); miotics (e.g. acetylcholine, carbachol, pilocarpine, physostigmine, isoflurophate, echothiophate, and pralidoxime); mydriatics (e.g., epinephrine, and phenylephrine); mydriatics/cycloplegics (e.g. atropine, scopolamine, cyclopentolate, tropicamide, and oxyphenonium); prostaglandins (e.g., cloprostenol, dinoprost tromethamine, fenprostalene, and fluprostenol); muscle relaxants (e.g., aminopentamide, chlorphenesin carbamate, methocarbamol, phenazopyridine, and tiletamine); smooth muscle stimulants (e.g., neostigmine, oxytocin, and propantheline); serotonin; urinary acidifiers (e.g., ammonium chloride, ascorbic acid, and methionine); vitamins/minerals (e.g., Vitamins A, B, C, D, K, and E); and other antioxidants, polyphenols (e.g., resveratrol).

Embodiments of the invention are also concerned with methods of producing polymer microparticles with active agents uniformly distributed throughout the polymer matrix and of isolating the microparticles containing the encapsulated agents which conform to a uniform, desirable size via coacervation. The particle size and composition are constructed in such a manner to facilitate mucoadhesion of the particles in the GI tract, but resist their absorption by the epithelial lining of the GI tract (i.e., release of the active agent payload in the small intestine, without the particle itself crossing the mucosal lining). In general, one technique involves mixing the active agent with the polymer, followed by generation of droplets of the desired size of the mixture. In one aspect, the technique involves rapid freezing of the droplets. In one aspect, the droplets are then crosslinked, such as by contacting the droplets with a crosslinking agent.

In one or more embodiments, an active agent suspension is first prepared by dispersing the active agent in a suitable aqueous- or oil-based carrier to facilitate even or homogenous distribution of the active agent throughout the polymer suspension prior to droplet formation. For example, a typical antibiotic, e.g., cephalosporin, can be mixed with Tween 20® and a small added amount of glycerin to facilitate its incorporation into the polymer matrix. Other types of active agents can be mixed directly with the polymer suspension.

The active agent suspension can further comprise pharmaceutically acceptable additives, such as solid or liquid fillers or diluents. Examples of such additives include acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials such as known to persons of ordinary skill in the art. A tabulation of ingredients listed by the above categories can be found in the U.S. Pharmacopeia National Formulary, 1990, pp. 1857-1859 which is incorporated herein by reference in its entirety.

Some examples of the materials which can serve as pharmaceutically acceptable carriers include but are not limited to sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sunflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; pyrogen-free water; isotonic saline; Ringer's solution, ethyl alcohol and phosphate buffer solutions, as well as other non-toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. The amount of active ingredient that can be combined with the carrier materials to produce a single dosage form will vary depending upon the particular dosage format. As used herein, the term “pharmaceutically-acceptable” means not biologically or otherwise undesirable, in that it can be administered to a subject, cells, or tissue, without excessive toxicity, irritation, or allergic response, and does not cause undesirable biological effects or interact in a deleterious manner with the other constituents of the composition in which it is contained. Pharmaceutically-acceptable ingredients include those acceptable for veterinary use as well as human pharmaceutical use.

The active agent suspension can then be encapsulated by the polymer matrix by any of a number of possible processes such as, but not limited to, those described herein.

In one or more embodiments, the active agent is combined with the polymer (and delivery enhancing moiety). The resulting mixture can then be vortexed or sonicated or otherwise combined for some period of time at a temperature ranging from about 4° C. to about 28° C. A stabilizer such as a gum (such as xanthan gum), may be added. Excipients to modify the time-release profile can also be added to this mixture. The polymer matrix is then crosslinked to yield the active agent encapsulated in the crosslinked polymer matrix (along with delivery enhancing moieties, and time-release excipients, if present).

More particularly, the mixture of active agent and polymer is extruded or dispensed from a suitable apparatus for forming droplets. Any suitable apparatus can be used, and will generally comprise a chamber for holding the active agent and polymer mixture, with the chamber being in fluid communication with a fluid passage that terminates in a dispensing outlet or tip. The dispensing tip will have an orifice through which the mixture is expelled as a droplet (aka microdroplet). The technique can be executed using a simple apparatus, such as a syringe and needle, as well as machines specifically designed for droplet generation. The desired size of the droplet can be controlled based upon the cross-sectional dimension of the orifice and the viscosity of the mixture. A spray nozzle for generating several droplets simultaneously can also be used. The invention is particularly suited for droplets having a surface-to-surface dimension (i.e., in the case of a spherical droplet, its diameter) of from 1 μm to about 1000 μm.

The droplets are then crosslinked to yield individual microparticles, such as by contacting the droplets with a suitable crosslinking agent, exposure to activating radiation or heat, or changing the pH, depending upon the selected polymer matrix. In one or more embodiments, the generated droplets are added dropwise to a solution of crosslinking agent, which in general will comprise an agitated or stirring bath of an effective amount of crosslinking agent dispersed in a solvent system. In one or more embodiments, an effective amount of time-release excipient can also be included in the crosslinking bath.

In one or more embodiments, the microparticles are formed by coacervation of the polymer. In particular, a polymer suspension containing the swellable and/or biodegradable polymer, delivery enhancing moiety, optional time-release excipients, and active agent can be formed into droplets which are then crosslinked to generate discrete particles (self-sustaining bodies). In some embodiments, the droplets are added to a bath containing the crosslinking agent (and optional time-release excipient) for coacervation and ionic gelation of the particles in the bath. In one or more embodiments, the generated droplets are rapidly frozen after generation, prior to initiating crosslinking. For example, as described in more detail below, the generated droplets can be contacted with liquid nitrogen to rapidly freeze the polymer suspension into droplet form, prior to crosslinking.

It will be appreciated that the particular crosslinking conditions can be selected depending upon the particular polymer system selected for the matrix. It will be understood that the various reagents will be pharmaceutically acceptable, and selected to minimize any adverse effects on the active agent. The resulting particles are individual/discrete self-sustaining bodies that generally comprise the crosslinked polymer matrix in a self-sustaining particulate form, with the delivery enhancing moiety and active agent substantially uniformly distributed throughout the crosslinked polymer matrix. In preferred embodiments, the delivery enhancing moiety is continuously presented on the exterior surface of the crosslinked polymer matrix/particle regardless of any polymer degradation. Preferably, the delivery enhancing moieties are present on the external surface of the particle, and orientated such that their functional groups can interact with the GI's epithelial surfaces, enterocytes, and their receptors to enhance the mucoadhesive property of the particles and facilitate absorption of the particle's active agent.

In one or more embodiments, the polymer suspension comprises sodium alginate (or potassium alginate) as the polymer base building block which is then, in at least one embodiment, converted to a more stable (crosslinked) form through ionic exchange. Fabrication of alginate particles which are employed as delivery vehicles of therapeutics includes gelation and crosslinking of the alginate matrix. Gel formation of alginate materials is obtained either by lowering the pH or by inducing nucleation with a divalent cation such as calcium ions (Ca⁺²) which cross-links pairs of guluronic acid units within the alginate polymer structure. Divalent cations can be used for crosslinking of sodium or potassium alginate. Suitable divalent cations for use in the invention include any ions capable of forming a gel upon interaction with alginate. More particularly, the divalent cations are preferably biocompatible. In one or more embodiments, the divalent cations are selected from the group consisting of calcium, barium, strontium, and combinations thereof. Advantageously, the divalent cation reacts with the alginate to yield a microparticle for each droplet comprising an alginate matrix in which the active agent, delivery enhancing moieties, and optional time-release excipients, if present, are entrapped. For example, upon contacting sodium or potassium alginate droplets with an aqueous solution of calcium chloride (e.g., 1% to 40% by weight, preferably 1-24% or any effective concentration) or calcium acetate, for example, the sodium (or potassium) of the alginate droplet is replaced by calcium. This reaction occurs rapidly at room temperature (e.g., 20-25° C.) or below resulting in the formation of helix-helix loaded aggregates which rapidly separate from the aqueous medium in the form of a coacervated precipitate.

Regardless of the embodiment, the resulting particle can then be dried to a moisture content of from about 1 to about 15%, and preferably from about 3 to about 8%. The resulting particles can also be freeze-dried and/or lyophilized for storage.

In addition to converting loaded aggregates to a water-insoluble form, calcium plays another key role in the molecular configuration of the oral dosage form. In particular, it causes cross-linkage of neighboring polymer molecules through calcium cross-linking. The resulting stability of the delivery system is set in a three-dimensional substantially-spherical configuration which serves not only to hold active agents more securely, but in the protection of the active agent from oxidative degradation, UV degradation, moisture degradation in addition to a vast number of other environmental stresses.

During crosslinking, the exchange of sodium (or potassium) by calcium particularly enhances multiple cross-linkage formation between subunit molecules of the polymer enabling precipitation. In embodiments utilizing alginate as the polymer, the alginate portion may range from about 5% to about 99.5% by weight in the final particle, preferably from about 50% to about 90%, and more preferably from about 65% to about 80%, while the other gums or copolymers (e.g., carrageenan, agar-agar, guar gum, xanthan gum, and/or chitosan) may optionally make up to about 20%, and preferably from about 0.1-5% of the final particle. In other embodiments, the sodium (or potassium) alginate is left in a gel form rather than in a precipitated form. The sodium, potassium, or calcium alginate polymer used, as above, may have a molecular weight ranging from 10,000-600,000 Daltons, or preferably 100,000-400,000 Daltons, or more preferably 300,000-320,000 Daltons, and still more preferably 305,000 Daltons.

It will be appreciated that the preparation of alginate particles on a uniform micron scale has been challenging. The particles tend to aggregate in the CaCl₂ bath during gelation, thus forming larger aggregates resulting in a non-uniform size distribution of the resulting gel particles. In addition, when particles are fabricated in this manner in order to encapsulate a therapeutic, the distribution of the therapeutic material becomes non-uniform within the alginate capsules.

Herein, a method has been designed to obtain crosslinked polymer microparticles in any range including, but not limited to, exemplary particles of 1 m to about 1000 m in diameter. The approach described in more detail below can yield microparticles with a uniform particle size distribution. In other words, the population of prepared microparticles has a narrow particle size distribution, which means that the majority (i.e., greater than about 75%) of the particles formed will be within +/−5 μm of the targeted particle size. For example, in the presented example, particles were prepared with a particle size ranging from 3 to 5 μm (i.e., +/−1 μm from a target size of 4 μm). These particles also contain uniformly distributed active agents within the polymer matrix, along with delivery enhancing moieties, and time-release excipients, if present. In addition, these particles have been engineered for controlled release of the active agents from the particles depending on the addition of excipients, such as mannitol and/or chitosan, into the formulation.

Some embodiments of the inventive method involve rapid freezing of the liquid droplets after droplet formation, but prior to crosslinking. For example, the methods include spraying a polymer suspension containing the active agent onto liquid nitrogen with a 120 kHz ultrasonic nozzle which produces frozen droplets (aka microdroplets) with the desired dimensions (e.g., 30×50 μm). It will be appreciated that other embodiments produce different sized droplets dependent upon the desired product size and the capabilities of the nozzle(s) used.

The frozen droplets are then contacted with an appropriate crosslinking agent to crosslink the polymer matrix. For example, in the case of alginate, frozen alginate microdroplets are formed by spraying an alginate suspension containing the active agent, delivery enhancing moieties, and time-release excipients, if present, onto liquid nitrogen. The resulting frozen microdroplets are then collected (e.g., with a strainer, spoon, etc.) and transferred to a CaCl₂ bath, where they begin to crosslink as they thaw due to the cross-linking of the alginate polymer in the individual droplet with Ca⁺² ions thus forming crosslinked microparticles. In other words, the thawing and crosslinking process occurs simultaneously or near simultaneously, and proceeds from the outside-in as the Ca⁺² ions infiltrate the frozen alginate droplet during thawing. Coacervates are formed as a result of electrostatic interaction between two aqueous phases during thawing, followed by ionic gelation as the material transitions from liquid to gel due to ionic interaction conditions at room temperature.

As the microdroplets defrost and thaw, the surface of the thawing droplet comes in contact with the crosslinking agent (i.e., in the case of alginate, Ca⁺² ions) and as these crosslinking agents reach the polymer within the droplet, the gelation and crosslinking processes occur within each droplet, proceeding from the surface to the interior. The degree of coacervation is inversely related to cross-sectional dimensions (i.e., diameter) of the resulting product as intermolecular forces drive out water molecules. In one or more embodiments, the droplets remain in contact with the crosslinking agent for a period of time sufficient to completely cure/crosslink the entire droplet from the surface to the core. In one or more embodiments, the droplets are stirred in the crosslinking bath for a period of about 4 to about 6 hours. The inventive method prevents aggregation of droplets during coacervation, preventing the fusion of droplets that would increase the overall size or agglomeration of the particles.

After the particles are crosslinked, they are removed from the crosslinking bath by centrifugation and washed with an aqueous solution several times (e.g., water). Alternatively, the crosslinked microparticles can be washed with an aqueous solution containing additional time-release excipient in embodiments concerned with faster-release particles.

Depending on the concentration of polymer in the suspension, different particle size batches can be obtained. In one embodiment, an ultrasonic nozzle can produce droplets with a size of 30×50 μm—and the resulting particles in suspension can be as large as large as 20 μm and as small as 3 μm. Atmospheric Spray Freeze Drying (ASFD) or lyophilization can then be used to convert the particles to a dry powder.

The same method can be used to produce larger or smaller size polymer particles by using a single fluid nozzle, or two fluid nozzle, which is calibrated to produce the desired size droplets.

The major advantages of the method are founded on obtaining uniform particle size distribution per batch prepared, including, but not limited to, batches of particles with a particle size of from about 3 μm to about 5 μm, as in the presented example. Further, these particles contain substantially uniformly distributed active agents within each particle body. As used herein, references to the “particle size” refer to the maximum surface-to-surface dimension of a given particle, such as the diameter in the case of substantially spherical particles.

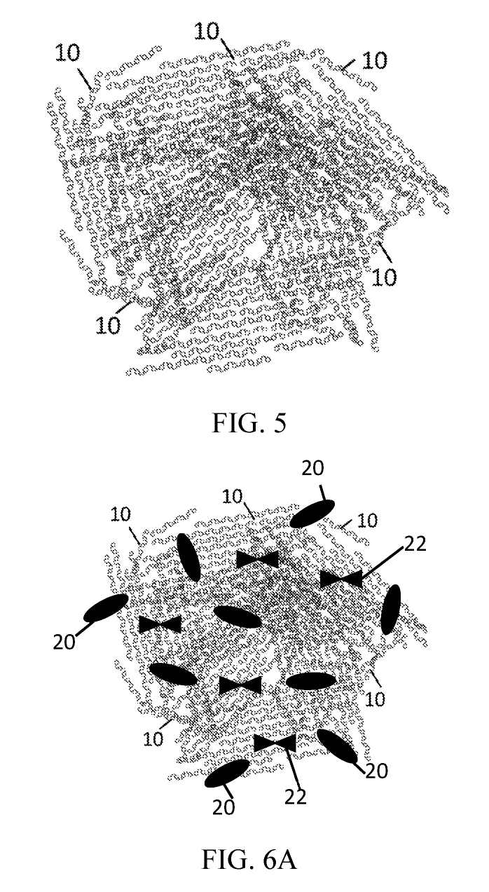

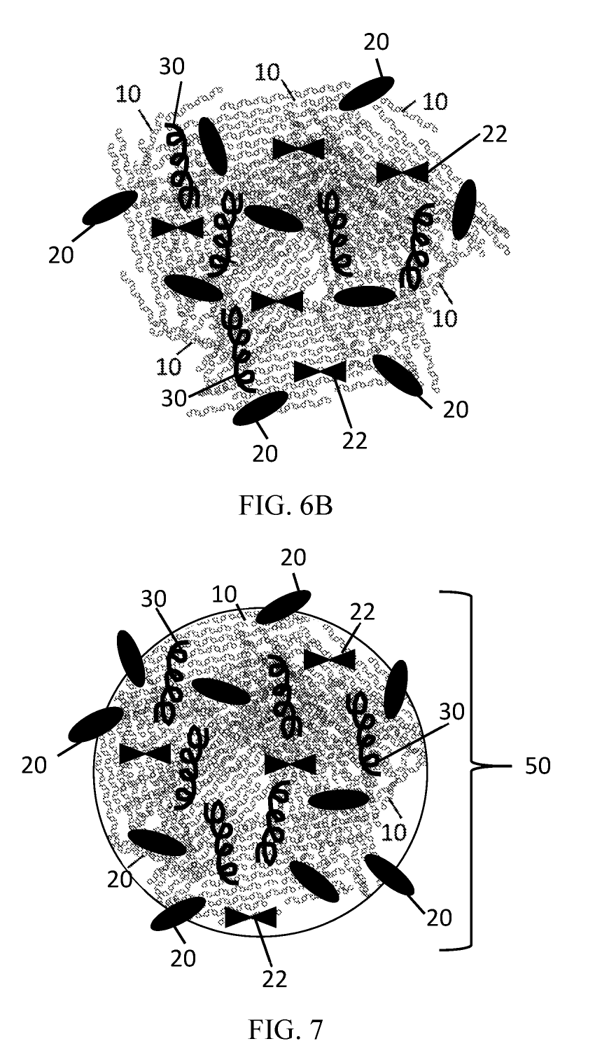

With respect to an exemplary embodiment, FIG. 5 shows a representation of a polymer matrix comprising alginate molecules 10. Shown in FIG. 6A is a representation of an aqueous alginate 10 suspension after it has been mixed with delivery enhancing moieties 20 to form an enhanced alginate mixture. The delivery enhancing moieties 20 become attached to or otherwise associated with the alginate molecules 10, such as through covalent, ionic, or physical interactions, as discussed herein. The delivery enhancing moieties 20 are entrapped within the matrix, as well as being presented on and extending from the particle surface once formed. Various additives can be included in this suspension, including time-release excipients 22, as shown in FIG. 6B. The delivery enhanced polymer is then combined and mixed with the active agent 30 desired to be encapsulated to yield the mixture for droplet generation. The mixture is then crosslinked to yield a microparticle 50, as depicted in FIG. 7, wherein the particles 50 have delivery enhancing moieties 20 presented on the surface and throughout the crosslinked polymer matrix (along with the active agent 30, and optional time-release excipient 22).

In one embodiment, the resulting crosslinked microparticles present a characteristic release of an active agent when these microparticles are exposed to the basic aqueous environment of the small intestine, which causes the swelling of the microparticles and allows the active agent to diffuse out of the polymer. Such microparticles are prepared according to the procedures described above, but without an accelerating time-release excipient. In an alternative embodiment, crosslinked microparticles present a characteristic faster release of an active agent when these microparticles are prepared from a suspension of polymer and an active agent in the presence of an accelerating time-release excipient, such as mannitol. The disclosed methods are capable of producing particles with a particle size of greater than 1 μm, preferably from 1 μm to about 1000 μm, more preferably from 1 m to about 200 μm, and even more preferably from about 1 m to about 20 μm.

The resulting microparticles have a structure characterized by the polymer chains crosslinked into a 3-dimensional network (i.e., the polymer matrix), with a porous structure and open and closed cells, in which the active agent and other constituents can be entrapped. The polymer matrix is characterized by this semi-rigid network that is permeable to liquids and gases, but which exhibits no flow and retains its integrity in the steady state. In other words, the microparticles are each self-sustaining bodies. The term “self-sustaining body” means that the polymer matrix, once formed, retains its shape without an external support structure, and is not susceptible to deformation merely due to its own internal forces or weight. The self-sustaining body is not pliable, permanently deformable, or flowable, like a jelly, putty, or paste, but is resilient, such that the matrix body may temporarily yield or deform under force. In other words, the self-sustaining body will recoil or spring back into shape after minor compression and/or flexing—it being appreciated that the polymer matrix will crack, break, or shear under sufficient exertion of external pressure or force. The resulting polymer microparticles are thus, a matrix-type capsule, meaning that they hold the encapsulated material throughout the volume of the particle body, rather than having a distinct shell as in a core-shell type capsule.

As discussed herein, the microparticles are resistant to enteric degradation. The particle body will not significantly degrade in stomach acids, and will localize in the small intestine. Without wishing to be bound by theory, the microparticle temporarily adheres to the epithelial lining of the small intestine facilitated by the mucoadhesive properties of the polymer matrix and the delivery enhancing moieties, which are oriented on the particle surface such that their functional groups can interact with the epithelia lining. In one or more embodiments, exposure to the acidic pH environment of the stomach will initiate an ion exchange in the polymer matrix, weakening the 3-dimensional structure. In one or more embodiments, subsequent exposure to basic pH environment of the small intestine causes the microparticle to swell and expand as the chains of the polymer matrix repel one another. This “opening” of the polymer matrix increases its mucoadhesive properties and releases the active agent directly in the vicinity of the lining of the small intestine significantly increasing the efficiency and efficacy of the uptake of the active agent into the blood stream. In one or more embodiments, the delivery enhancing moieties further enhance this uptake process. The polymer matrix itself is then cleared and excreted through normal GI mechanisms.

In one or more embodiments, the crosslinked microparticles can be dispersed into a second polymer formulation containing alginate or other selected polymer followed by the spraying and coacervation protocols described above for the purpose of forming a second polymer layer around the original self-sustaining particles. This second polymer formulation employed can contain additional active agents, such as the one contained in the original self-sustaining particles, or new active agents chosen to enhance or complement the original agent; the polymer used can be pristine or modified as above with additional targeting moieties, in order to protect the original active ingredient and/or to interact with additional receptors throughout the delivery route. In some embodiments, formulations contain additional additives to regulate the time-release profile of each layer separately or in conjunction.

Still other embodiments repeat the layering method to create multi-layered particles with compartmentalized formulations. The resulting multi-layer particle(s) can be used as part of an oral dosage form for administering the active agent to a subject. In preferred embodiments of the presently claimed and disclosed inventive concept(s), the polymer matrix consists essentially (and even consists) of calcium alginate (obtained for example by reaction of sodium alginate with a calcium salt such as CaCl₂). In the configuration contemplated herein it forms multiple cross-linked helix-helix aggregates resulting in superior encapsulation strength. In alternative embodiments, a minor portion of the matrix may further comprise quantities of one or more other natural gums for cross-linking and thickening, such as, but not limited to, carrageenan, agar-agar, guar gum, and/or xanthan gum.

The resulting delivery enhanced particles may then be packaged within a number of suitable containment systems for shelf storage prior to the administration to the subject.

In one or more embodiments, the microparticles are used for delivery of an active agent to a subject. In one or more embodiments, the enhanced or modified microparticles can be used in a unit dosage form for oral administration of the active agent to a subject. The term “unit dosage form” refers to a physically discrete unit suitable as a unitary dosage for human or animal use. Each unit dosage form may contain a predetermined amount of the microparticles (and/or other active agents) in a carrier calculated to produce the desired effect.

A protease inhibitor may be included in the dosage form contemplated herein as well. Examples of such protease inhibitors include, but are not limited to, AEBSF-HCI, Amastatin-HCI, (epsilon)-Aminocaproic acid, (alpha)1-Antichymotrypsin from human plasma, Antipain-HCL, Antithrombin III from human plasma, (alpha)1-Antitrypsin from human plasma, (4-Amidinophenyl-methane sulfonyl-fluoride), Aprotinin, Arphamenine A, Arphamenine B, Benzamidine-HCI, Bestatin-HCI, CA-074, CA-074-Me, Calpain Inhibitor I, Calpain Inhibitor II, Cathepsin Inhibitor Z-Phe-Giy-NHO-Bz-pMe, Chymostatin, DFP (Diisopropylfluoro-phosphate), Dipeptidylpeptidase IV Inhibitor, H-Giu-(NHO-Bz)Pyr, Diprotin A, E-64, E-64d (EST), Ebelactone A, Ebelactone B, EDTA-Na2, EGTA, Elastatinal, Hirudin, Leuhistin, Leupeptin-hemisulfate, (alpha)2-Macroglobulin from human plasma, 4-(2-Aminoethyl)-benzenesulfonyl fluoride hydrochloride, Pepstatin A, Phebestin, Phenyl methyl sulfonyl fluoride, Phosphoramidon, (1-Chloro-3-tosylamido-7-amino-2-heptanone HCI, (1-Chloro-3-tosylamido-4-phenyl-2-butanone), Trypsin inhibitor from egg white (Ovomucoid), and Trypsin inhibitor from soybean.

In practice, the microparticles are administered to a subject in need thereof. Oral administration routes are particularly preferred according to embodiments of the invention; however, that does not necessarily preclude other modes of administration. In one or more embodiments, treatment methods comprise administering microparticles comprising a therapeutically-effective amount of active agent to the subject. As used herein, a “therapeutically effective” amount refers to the amount of the active agent that will elicit the biological or medical response of a tissue, system, animal, or human that is being sought by a researcher or clinician, and in particular elicit some desired therapeutic effect. One of skill in the art recognizes that an amount may be considered therapeutically effective even if the condition is not totally eradicated but improved partially. The microparticles are advantageously, resistant to gastric fluid (i.e., enterically resistant), but allows the particles to swell and release active agents at conditions consistent with the environment of the targeted region of the GI tract.

Depending upon the active agent and condition involved, a single dose may provide adequate therapeutic effect. In one or more embodiments, additional dosages can be administered, by the same or different route, to achieve the desired prophylactic or therapeutic effect. The microparticles can also be administered using a prime and boost regime if deemed necessary. In one or more embodiments, the microparticles are administered to the subject as part of ongoing treatment, such as in the case of a daily treatment regimen. Regardless of the embodiment, once administered, the microparticles preferably localize in the GI tract, temporarily adhere to the intraluminal surface of the lining of the GI tract, release their active agent payload which enters the blood stream, and then the remainder of the particles themselves are excreted. In other words, the microparticles according to the various embodiments of the invention do not themselves cross the intestinal mucosa, and are not capable of transmucosal passage across the intestinal mucosa into the intestinal bloodstream. Rather, the polymer matrix is degraded and/or swelled in the GI tract, releasing its active agent, which is selectively taken up into the intestinal bloodstream (without the rest of the particle).

Additional advantages of the various embodiments of the invention will be apparent to those skilled in the art upon review of the disclosure herein and the working examples below. Another aspect of the invention includes particle enhancements, such as to optimize particle size and/or solid dispersion. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

This example describes the modification of alginate with retinyl glycinate moieties to obtain a product incorporating carotenoid units as depicted in FIG. 2.

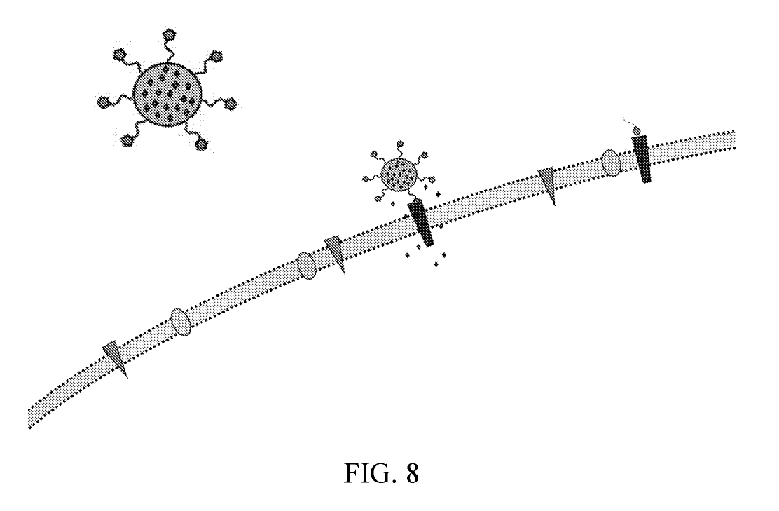

Modified alginate polymers attached to retinyl glycinate moieties are used to facilitate the physical attachment of these particles onto cells of the GI tract. Retinol (Vitamin A), of which retinyl glycinate is a modification, is a substance typically obtained through diet, for which there exist numerous receptors on cells of the GI. Polymers possessing these moieties are readily recognized via the interaction of retinol with its receptor and bind to mucosal cells of the intestine, allowing for minimized travel distances and increased dwell time for API elution from the polymer matrix to the GI absorptive surface. (See FIG. 8).

The initial step in covalently attaching retinol units to the alginate polymer involves the linking of glycine to retinol by an ester bond. This reaction is mediated through EDCI and DMAP in anhydrous tetrahydrofuran (THF) under nitrogen. The amino group of the glycine molecules is prevented from reacting by an Fmoc-protecting group. After the reaction is complete, the Fmoc-group is removed with piperidine, the reaction mixture is acidified with acetic acid, and the solvent is removed under nitrogen. Subsequently, the amine unit of the glycine linker is coupled to the alginate using standard amide coupling reagents such as EDCI and NHS in THF/aqueous media.

Retinol (400 mg, 1.4 mmol), Fmoc-glycine (420 mg, 1.4 mmol) and DMAP (35 mg, 0.28 mmol) were placed in a dry round bottom flask under nitrogen. Then, 20 mL of dry THF were added and the solution was cooled to 0° C. while stirring. At this point, EDCI (540 mg, 2.8 mmol) in 40 mL of THF was added by cannula. The resulting reaction mixture was allowed to warm to room temperature and stirred for 18 h. Subsequently, a degassed aqueous solution of sodium ascorbate (0.5 mL, 1.5 M, 0.8 mmol) followed by piperidine (1.0 mL, 10 mmol) were added, and the reaction mixture was stirred for 30 min. Acetic acid (1.0 mL, 18 mmol) was then added, and the solvent was evaporated under nitrogen down to 5 mL. The resulting residue was resuspended in 20 mL of dimethyl sulfoxide (DMSO) and used in the next step without purification. Progress of the reactions was followed by thin layer chromatography.

The modification of alginate with retinol-glycinate units was accomplished by adding EDCI (1.0 g, 5.2 mmol) and NHS (0.3 g, 2.6 mmol) to a dispersion of alginate (1.0 g) in 150 mL of distilled water. After stirring for 2 hours, the dispersion was degassed with nitrogen. Then, the DMSO suspension of the previous reaction was injected into the alginate suspension, and the mixture was left to stir overnight at room temperature. Unreacted retinol was extracted with ethyl acetate (EtOAc), and the modified alginate was precipitated with a mixture of THF and acetonitrile (ACN). The obtained solid (Retinol-AA) was dried under nitrogen, and further purification was accomplished by dialysis (10 K MWCO membrane) followed by lyophilization. The obtained retinol-alginate (retinol-AA) solid was characterized by IR: Stretch at 3019, 3047, and 3066 cm⁻¹ (C═C stretch) and at 1692 cm⁻¹ (C═O amide stretch). These bands are not present in the IR spectra of non-modified alginate. Also the presence of retinol ester was detected by a colorimetric assay with TFA which resulted in a blue color which faded in a 10 min period. In addition, the presence of retinol ester was verified by the fluorescence (blue-green) property of the modified alginate when irradiated at 365 nm.

### Example 2

### Coacervation of Insulin in Retinol-Alginate (Retinol-AA)

Two formulations were employed to prepare particles of retinol-alginate encapsulating insulin. One was made entirely of the modified alginate prepared in example 1 while the other also contained mannitol.

In the first embodiment, Retinol-AA/Insulin formulations were prepared by dispersing 200 mg of Retinol-AA from example 1 in 50 mL of distilled water. Then, a solution of insulin (100 mg) in 1% acetic acid (5 mL) was prepared, and added dropwise to the retinol-AA dispersion while stirring followed by 5 mL of additional 1% acetic acid. The dispersion was stirred for 30 min before adding more distilled water to bring the volume to 125 mL.

In the second embodiment, Retinol-AA-mannitol/Insulin formulations were prepared in a similar manner; 200 mg of retinol-AA from example 1 was dispersed in 50 mL of distilled water. Then, a solution of mannitol (600 mg) in 5 mL of 1% acetic acid was added to a solution of insulin (100 mg) in acetic acid (5 mL, 1% in water). Subsequently, the insulin/mannitol solution was added dropwise to the dispersion while stirring. The mixture was stirred for 30 min before adding more distilled water to bring the volume to 125 mL.

The formation of the coacervated retinol-AA particles involved the spraying the formulation onto liquid nitrogen (N₂) with an ultrasonic nozzle to generate frozen microdroplets. These frozen microdroplets were transferred into an aqueous CaCl₂ solution for first embodiment, or an aqueous CaCl₂/mannitol solutions for the second embodiment to allow for coacervation to occur on each individual droplet. The particles were removed from the CaCl₂ baths and washed, with water for the first embodiment or an aqueous mannitol solution (6 mg/mL) for the second embodiment, by centrifugation, three times. The particles were finally dried by lyophilization or Atmospheric Spray Freeze Drying.

Next, the powders, CaCl₂ baths, and aqueous washes from each formulation were assayed for insulin content by High Pressure Liquid Chromatography (HPLC) with an Agilent 1260 HPLC equipped with a binary pump, degasser, autoinjector, and diode array detector. The HPLC runs were carried out in a gradient mode at a flow rate of 1 mL/min and detection at 214 nm using a Poroshell 300SB-C18 (2.1×75 mm, 5 μm, Agilent). The Mobile phases consisted of 95% water, 5% (ACN), 0.075% trifluoroacetic acid (TFA) v/v (Mobile Phase A), and 5% water, 95% ACN, 0.075% TFA, v/v (Mobile Phase B). The method was held at 20% B for 1 min at the start. Then, a gradient was applied from 20% B to 60% B in 6 min, and held at 60% B for 0.5 min. Next, the gradient was reset to 20% in 0.5 min and re-equilibrated for 2 min. Under these conditions, insulin eluted at about 3.2 min (% RSD=0.07).

Calibration curves, which measure Peak Area vs Insulin Concentration, as well as Peak Height vs Insulin Concentration, were conducted every time insulin content was assayed by HPLC methodology. To conduct these calibration curves, a 1 mg/mL standard stock solution of insulin in 0.01N HCl was prepared and kept stored at 4° C. A working stock solution at a concentration of 0.1 mg/mL was made from the stock solution, and nine standard solutions, ranging in concentration from 3.5 to 60 μg/mL in 0.01 M HCl were prepared by a serial dilution from the Working Stock.

### Example 3

In order to assess the capacity of these particles to release their active agent as a function of the formulation, powder samples were assayed in triplicate in the different media. The insulin content in media after incubation with the retinol-AA and retinol-AA-mannitol powder formulation was assessed after 3 hrs incubation by HPLC according to the method described above. The concentration of insulin remaining in the media is presented in Table 1 below.

TABLE 1
Insulin found in the indicated media, by HPLC, after
incubation with particles prepared by the indicated
formulations for 3 hours at 37° C., 100 rpm.

| Formulation | 0.2M NaHCO3 | 0.01M HCl |
|---|---|---|
| Retinol-AA | 32% | 0% |
| (mg Insulin/mg Solid) | [0.068 + 0.007] | [0.000] |
| Retinol-AA-mannitol | 99% | 0% |
| (mg Insulin/mg Solid) | [0.054 + 0.004] | [0.000] |

The results obtained after incubating particles in an HCl (pH 2) solution indicate that there is no insulin released into media and that the particles remain coacervated thus protecting the insulin in acidic media. In the basic solution (NaHCO₃, pH 8.3), the particles open up to release entrapped insulin.

The suspensions of particles in Simulated intestinal fluid (SIF) (without pancreatin, pH 7.4-7.6, Ricca, USP XXII Formulation) were incubated at 37° C. for a maximum of 4 hours before the media was subjected to HPLC as described above to quantitate the material released from the particles. Table 2 illustrates the fraction of active agent (insulin) released into media.

TABLE 2
Content of insulin released, assayed by HPLC, after incubating
the particles in SIF for 4 hours at 37° C., 100 rpm.

| Formulation | 4 hour |
|---|---|
| Retinol-AA | 33% ± 1% |
| Retinol-AA-mannitol | 90% + 5% |

The formulation without mannitol displayed slow release upon reconstitution while the one containing mannitol displayed faster release of the active. These data illustrate the variability of release time for active ingredient depending on formulation of the modified alginate and represent examples of the ability to tune the release via formulation.

### Example 4

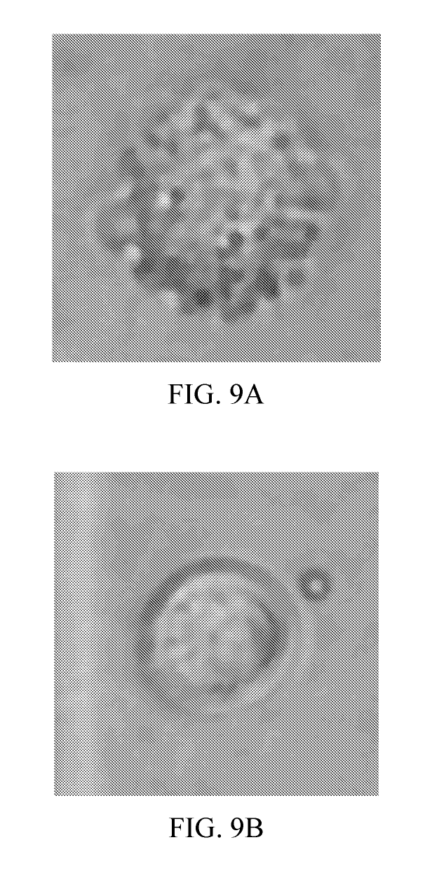

In order to demonstrate the efficacy of incorporating delivery enhancing molecule into the alginate in adsorbing particles to target cells, alginate particles containing retinol as the delivery enhancing molecule retinol, as illustrated in FIG. 2, were incubated in culture with the soluble rat enterocyte line IEC-6 (ATCC® CRL-1592™). Cells incubated with the inventive particles are illustrated in the micrograph of FIG. 9a. Cells incubated with particles lacking the delivery enhancing moiety are illustrated in FIG. 9b as controls.

### Example 5

In vivo testing of Resveratrol-Containing Particles in Rodent Model Four different Resveratrol-containing particles were generated using the basic protocol from Example 1, except that the carotenoid moieties were physically mixed, but not covalently attached to the alginate polymer matrix. The formulations are outlined in Table 3 below.

TABLE 3
Resveratrol Formulations

| | | Average |
|---|---|---|
| Formulation | | Encapsulated Dose |
| Name | Description | (mg/kg) |
| TA-RU | Unencapsulated Resveratrol | NA |
| TA-R1 | Resveratrol Encapsulation 1 | 395 |
| TA-R2 | Resveratrol Encapsulation 2 | 355 |
| TA-R3 | Resveratrol Encapsulation 3 | 395 |

The Pharmacokinetics of the resveratrol-containing particles (and unencapsulated control) were assayed in Sprague-Dawley (SD) rats (Charles River Laboratories).

Animals were administered 20 mg/kg resveratrol p.o. Blood was collected at the indicated time points (1, 2, 3, and 4 hrs). Whole blood was processed to plasma and analyzed for resveratrol quantity.

Parameters were estimated using Watson pharmacokinetic software 7.4.2 (Thermo Electron Corporation) using a non-compartmental approach consistent with the various routes of administration. All parameters were generated from Insulin individual animal concentrations in plasma. Parameters were estimated using nominal sampling times to be relative to the start of each dose administration (within an acceptable tolerance limit). As no dose formulation samples were analyzed, the nominal calculated dose for active ingredient (20 mg/kg, as per the study protocol, was used for dose-normalization purposes).

The area under the test item concentration versus time curve (AUC) was calculated using the linear-log linear trapezoidal method. AUC was not calculated for PK profiles with less than 3 consecutive quantifiable concentrations of test article at separate time points. When practical, the terminal elimination phase of each concentration versus time curve was identified using at least the final three observed concentration values. The slope of the terminal elimination phase was determined using linear regression on the unweighted concentration data. Parameters relying on the determination of the terminal elimination phase were excluded from the summary statistics where the extrapolation of the AUC to infinity represented more than 40% of the total area and/or if the number of regression points were less than three. The parameters described below were reported to 3 significant figures.

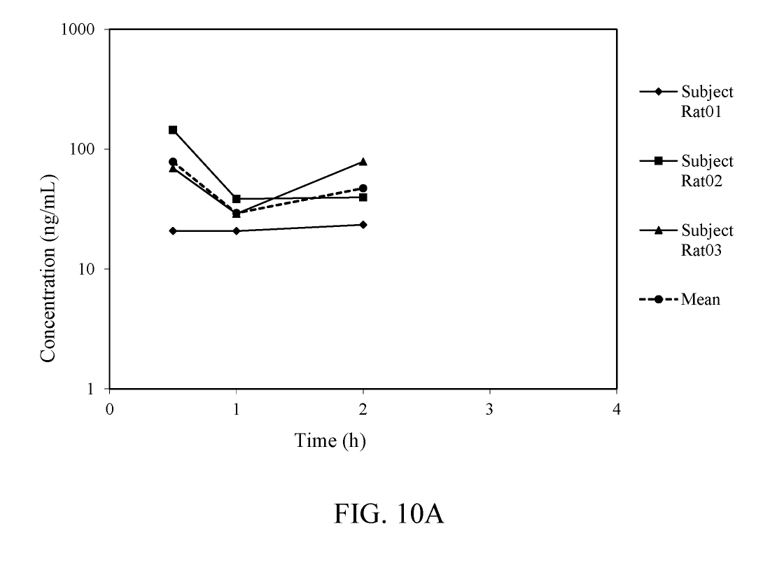

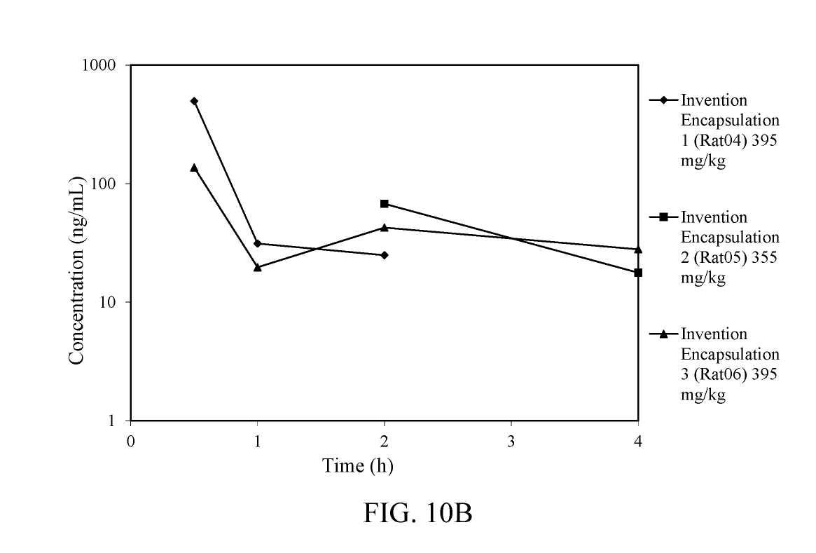

Animals were fasted overnight, food was returned 4 hours post dosing. Mean resveratrol concentration from the blood of animals was assessed following dosing of 20 mg/kg per animal p.o. As a control, unencapsulated material was Blood samples were taken over the next 24 hours and assayed at various time points to establish a reference curve for plasma concentration. These data for the TA-RU animals receiving unencapsulated active agent and 3 different encapsulations are presented in FIGS. 10A-B illustrating the appearance and steady release of agent into the blood.

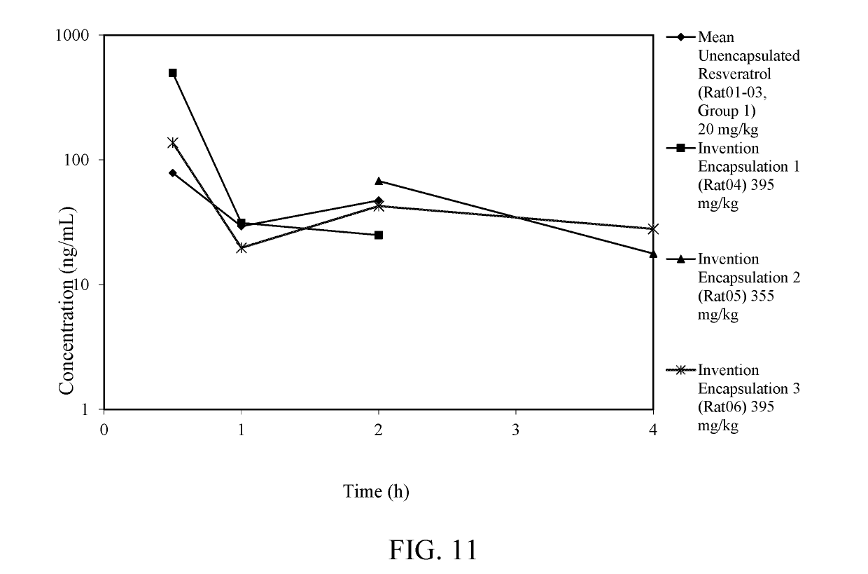

Three formulations of resveratrol-containing particles (TA-R1, TA-R2, and TA-R3) were dosed p.o. as indicated above. Concentrations of resveratrol over time are illustrated for each formulation. FIG. 11 shows blood resveratrol concentrations dosed per animal p.o. All groups show rapid infusions of resveratrol in the blood peaking at 0.5 hrs post-dosing. Aggregated data is summarized for individual animals in Table 4, below.

TABLE 4
Plasma Concentrations of Resveratrol (ng/mL) for the following preparations in Orally
Dosed Sprague-Dawley Rats

| | | Mean | | | |
|---|---|---|---|---|---|
| | | Unencapsulated | | | |
| | | Resveratrol | | | |
| | | (Rat01-03, | Invention | Invention | Invention |
| | | Group 1) | Encapsulation 1 | Encapsulation | Encapsulation 3 |
| | Time | 20 mg/kg | (Rat04) 395 mg/kg | 2 (Rat05) 355 mg/kg | (Rat06) 395 mg/kg |
| | (h) | (ng/mL) | (ng/mL) | (ng/mL) | (ng/mL) |
| | 0 | NA | BQL < 10.0 | BQL < 10.0 | BQL < 10.0 |
| | 0.5 | 78.4 | 498 | BQL < 10.0 | 137 |
| | 1 | 29.3 | 31.2 | BQL < 10.0 | 19.7 |
| | 2 | 47.2 | 24.9 | 67.6 | 42.6 |
| | 4 | NA | BQL < 10.0 | 17.7 | 27.9 |
| | 6 | NA | 13.9 | BQL < 10.0 | BQL < 10.0 |
| | 8 | NA | BQL < 10.0 | BQL < 10.0 | BQL < 10.0 |
| | 12 | NA | BQL < 10.0 | BQL < 10.0 | BQL < 10.0 |
| | 24 | NA | BQL < 10.0 | BQL < 10.0 | BQL < 10.0 |
| AUC₍₀₋ₓ₎ | ng · h/mL | 82.8 | 312 | 108 | 165 |
| AUC_{(0-∞)} | ng · h/mL | NA | 400 | 134 | 297 |
| % AUC | % | NA | 22.0 | 19.4 | 44.4 |
| Extrap | | | | | |
| Cmax | ng/mL | 82.4 | 498 | 67.6 | 137 |
| tmax | hr | 1.50 | 0.500 | 2.00 | 0.500 |
| t½ | h | NA | 4.41 | 1.03 | 3.27 |

BQL = Below the Level of Quantitation
NA = Not Applicable

1. A microparticle for oral delivery of active agents to a subject, comprising a self-sustaining body having an exterior surface, said self-sustaining body comprising a crosslinked polymer matrix and an active agent encapsulated therein, wherein said active agent is distributed throughout said polymer matrix, said polymer matrix further comprising a plurality of delivery enhancing moieties, wherein at least a portion of said delivery enhancing moieties are presented on the exterior surface of said self-sustaining body, and wherein said microparticle is resistant to enteric degradation and will localize in the gastrointestinal tract of said subject without crossing the intestinal mucosa into the intestinal bloodstream, wherein said microparticle releases said active agent from said polymer matrix in the small intestine of said subject to thereby orally deliver said active agent to said subject.

2. The microparticle of claim 1, wherein said polymer matrix comprises a crosslinked polymer selected from the group consisting of polyvinyl alcohol, poly(ethylene glycol), polylactic acid, poly-L-lactic acid, polycaprolactone, polyglycolic acid, poly(lactic-co-glycolic acid), polyhydroxyalkanoate, poly [N-(2-hydroxypropyl) methacrylamide], hyaluronic acid, gelatin, cellulose derivatives, xanthan gum, chitosan, alginate, and pectin.

3. The microparticle of claim 1, wherein said delivery enhancing moieties are ionically, covalently, or supramolecularly bound to the polymer matrix.

4. The microparticle of claim 1, wherein said delivery enhancing moieties are physically entrapped within the polymer matrix.

5. The microparticle of claim 1, wherein said delivery enhancing moieties are selected from the group consisting of C₆-C₂₈ fatty acids, isoprenoids, vitamins, signal peptides, and combinations thereof.

6. The microparticle of claim 1, said microparticle further comprising one or more time-release excipients distributed throughout said polymer matrix.

7. (canceled)

8. The microparticle of claim 6, wherein said time-release excipients are selected from the group consisting of monosaccharides, disaccharides, sugar alcohols, polyethylene glycols (PEGs), Sorbitan, Tween, Polysorbate, poloxamers, chitosan, and combinations thereof.

9. The microparticle of claim 1, wherein said microparticle has a particle size of from 1 μm to about 1000 μm.

10. The microparticle of claim 1, wherein said active agent is selected from the group consisting of therapeutic and nutraceutical agents, such as antibiotics, antivirals, antioxidants, oncological agents, anti-lipids, antihypertensives, cardiac drugs, antidiabetic agents, vitamins, minerals, proteins, peptidomimics, microorganisms, monoclonal antibodies, and/or RNA or DNA molecules.

11. A composition comprising a therapeutically-effective amount of a plurality of microparticles according to claim 1 dispersed in a pharmaceutically acceptable carrier.

12. The composition of claim 11, wherein said composition is in unit dosage form suitable for oral administration.

13. (canceled)

14. The composition of claim 12, wherein said unit dosage form is selected from the group consisting of capsules, tablets, liquid, and powder.

15. A method of orally administering an active agent to a subject in need thereof, said method comprising orally administering a therapeutically effective amount of microparticles according to claim 1 to said subject.

16. (canceled)

17. A method of forming microparticles, said method comprising:
providing a polymer suspension, said polymer suspension comprising a polymer matrix precursor, delivery enhancing moieties, dispersed in a solvent system;
combining said polymer suspension with an active agent to yield a mixture;
crosslinking said polymer matrix precursor in said mixture to yield a crosslinked polymer matrix in the form of a self-sustaining body having an exterior surface, wherein said active agent is distributed throughout said polymer matrix, and wherein at least a portion of said delivery enhancing moieties are presented on the exterior surface of said self-sustaining body, said self-sustaining body being resistant to enteric degradation but capable of localization in the gastrointestinal tract of a subject without crossing the intestinal mucosa into the intestinal bloodstream.

18. The method of claim 17, wherein said crosslinking comprises:
adding said mixture dropwise to a solution of crosslinking agent to yield said self-sustaining body.

19. The method of claim 18, wherein said adding comprises generating droplets of said mixture and dropping said droplets into said solution of crosslinking agent.

20. The method of claim 19, further comprising rapidly freezing said droplets of said mixture to yield frozen droplets, before dropping said frozen droplets into said solution of crosslinking agent.

21. The method of claim 20, wherein said rapidly freezing comprises contacting said droplets with liquid nitrogen.

22. The method of claim 21, wherein said contacting comprises spraying droplets of said mixture onto said liquid nitrogen to yield said frozen droplets.

23. The method of claim 17, wherein said mixture further comprises one or more time-release excipients.
